(19) United States
(12) Patent Application Publication
(10) Pub. No.: US 2005/0143826 A1
(43) Pub. Date: Jun. 30, 2005

(54) Disk repair structures with anchors

(75) Inventors: James F. Zucherman, San Francisco, CA (US); Ken Y. Hsu, San Francisco, CA (US)

Correspondence Address: FLIESLER MEYER, LLP, FOUR EMBARCADERO CENTER, SUITE 400, SAN FRANCISCO, CA, 94111, US

(73) Assignee: St. Francis Medical Technologies, Inc., Alameda, CA (US)

(21) Appl. No.: 11/002,167

(22) Filed: Dec. 2, 2004

Related U.S. Application Data
(60) Provisional application No. 60/528,954, filed on Dec. 11, 2003.

(51) Int. Cl. A61F 2/44

(52) U.S. Cl. 623/17.16; 623/17.11

(57) ABSTRACT

The present invention is directed to a device that is used to repair an injury or defect in the anulus of the intervertebral disk. The implant is characterized by having a flexible structure that is anchored to the vertebral bone. The flexible structure is connected with a patch that the flexible structure sustains over the injury or defect.

### CLAIM OF PRIORITY

U.S. Provisional Patent Application 60/528,954 entitled DISK REPAIR STRUCTURES WITH ANCHORS, by James F. Zucherman et al., filed Dec. 11, 2003 (Attorney Docket No. KLYCD-05005US0).

### CROSS-REFERENCE TO RELATED APPLICATIONS

This application is related to the following U.S. patent application Ser. No. ______, entitled DISK REPAIR STRUCTURES FOR POSITIONING DISK REPAIR MATERIAL, filed XX/XX/04, Attorney Docket No. KLYCD-05005US3, concurrently with the instant application, and incorporated fully by reference.

### FIELD OF THE INVENTION

This invention relates to a vertebral disk repair implant and method.

### BACKGROUND OF THE INVENTION

The spinal column is a biomechanical structure composed primarily of ligaments, muscles, vertebrae and intervertebral disks. The biomechanical functions of the spine include: (1) support of the body, which involves the transfer of the weight and the bending movements of the head, trunk and arms to the pelvis and legs; (2) complex physiological motion between these parts; and (3) protection of the spinal cord and nerve roots.

The intervertebral disk plays an important role in the biomechanical structure of the spine. It cushions the vertebrae and allows for controlled motions of these bones. An intervertebral disk has two components: (1) the nucleus pulposus, or “nucleus”; and (2) the anulus fibrosis, or “anulus.” The disk is positioned between two vertebral endplates located between adjacent vertebrae.

Each endplate creates an intermediate zone between the flexible disk and the rigid bone of the vertebrae. An endplate consists of thin cartilage overlying a thin layer of hard cortical bone. The hard cortical bone of the endplate is connected with cancellous bone of the vertebrae, which is spongy and vascularized.

The anulus is a tough, fibrous ring that has 15-20 overlapping layers that together are resistant to torsion. The ring connects adjacent vertebrae. It also houses the nucleus pulposus.

The nucleus is a gel-like substance that is high in water content. It helps maintain the shape of the anulus without decreasing its flexibility. When a force acts upon adjacent vertebrae, the nucleus moves with the anulus.

Trauma or disease may displace or damage the spinal disk. A disk herniation occurs when the anulus fibers are weakened or torn and the nucleus becomes permanently bulged, distended, or extruded out of its normal space within the confines of the anulus. The herniated or so-called “slipped” nucleus can compress a spinal nerve, causing leg pain, loss of muscle control, or even paralysis. Also, as the disk degenerates, the nucleus loses its water binding ability and deflates, which decreases the height of the nucleus. In turn, because of the decrease in height, the anulus buckles. In regions of buckling of the anulus, either circumferential or radial anulus tears may occur, potentially resulting in persistent and disabling back pain. Back pain may be compounded by adjacent, ancillary spinal facet joints which are forced into an overriding position from the buckling of the anulus.

Degenerated, diseases, or traumatized disks prevent people from working and can severely impact the lives of patients and their families. The pain associated with such conditions often is treated with medication and/or surgery. Of course, it is desirable to eliminate the need for major surgery for all individuals, particularly the elderly. Therefore, an easily implantable prosthetic is needed for sealing and promoting healing of injuries or defects in the anulus to prevent recurrence of disk herniation, the resulting impingement of nerves, and other effects on the anatomy of the spine.

### BRIEF DESCRIPTION OF THE DRAWINGS

FIG. 1A is a perspective view of an embodiment of the invention including a prosthetic intervertebral disk implant where an anulus patch is not yet connected with the flexible wire structure.

FIG. 1B is a perspective view of a prosthetic intervertebral disk implant with the anulus patch fully connected with the trumpet or cone-shaped flexible wire structure.

FIG. 1C is a perspective view of a prosthetic disk implant with an anulus plug.

FIG. 2A is a side cut-away view of an embodiment of the invention including an implanted intervertebral disk implant, viewed along a sagittal plane, the implant anchored to the top vertebra of two adjacent vertebrae on either side of an injured or defective disk.

FIG. 2B is a side cut-away view of an embodiment of the invention including an implanted intervertebral disk implant, viewed along a sagittal plane, the implant anchored to the bottom vertebra of two adjacent vertebrae on either side of an injured or defective disk.

FIG. 3 is a perspective view of an embodiment of the invention including an intervertebral disk implant with a flexible wire structure made of mesh and having hooks at the open end that connects with the anulus patch.

FIGS. 4A and 4B are perspective views of embodiments of the invention including intervertebral disk implants. FIG. 4A depicts an implant with a flexible wire structure having a single branch of a plurality of wires that flares into a cone shape toward the end that connects with the anulus patch. FIG. 4B is similar to FIG. 4A, except that the cone shaped part of the flexible wire structure is a wire mesh or weave with wires running along an axis that is substantially perpendicular to the axis of the single branch.

FIG. 5 is a side cut-away view of an embodiment of the invention including an implanted intervertebral disk implant, viewed along a sagittal plane, the implant anchored to the bottom of two adjacent vertebrae on either side of an injured or defective disk, and having a flexible wire structure consisting of a single branch that connects with the anulus patch, the anulus patch then sutured onto the anulus around the injured or defective site.

FIG. 6 is a side cut-away view of an embodiment of the invention including two implanted intervertebral disk implants, viewed along a sagittal plane, with first implant anchored to the bottom of two adjacent vertebrae, and the second implant anchored to the top of the two adjacent vertebrae.

FIG. 7 is a side cut-away view of an embodiment of the invention including an implanted intervertebral disk implant, viewed along a sagittal plane, the implant anchored to the bottom of two adjacent vertebrae on either side of an injured or defective disk, and having a spiral-shaped flexible wire structure.

FIG. 8 is a perspective view of an embodiment of the invention including an implant having a spiral-shaped flexible wire structure showing that the structure can be made shorter to accommodate the anatomy of the intervertebral space by cutting at a point in between the first end and the second end of the flexible wire structure as, for example, where indicated.

FIG. 9 includes a flow chart of an embodiment of the implantation method of the invention.

FIGS. 10A and 10B include a side view of an embodiment of the invention being positioned through a cannula according to the method of the invention.

### DETAILED DESCRIPTION OF THE PREFERRED EMBODIMENTS OF THE INVENTION

Embodiments of the present invention relate to a prosthetic intervertebral spinal implant for repairing the anulus of an intervertebral disk. The implant also serves to cushion impact on the spine. Specifically, the embodiments of the present invention concern a flexible structure that can be anchored to a vertebral bone endplate at a first end, and that sustains in place an anulus patch over an injury or defect in the anulus of a disk. Embodiments of the disclosed invention have the added benefit of functioning like the nucleus pulposus material to cushion impact on the spine, prevent further herniation, prevent narrowing of the intervertebral disk space and destabilization of the spine, and promote effective repair and healing of the injured anulus.

Embodiments of the present invention include a prosthetic intervertebral disk implant for implantation to repair an injury or defect in the anulus and to prevent narrowing of the intervertebral disk space. The implant is positioned inside the intervertebral disk space, which is defined by the bone endplates of two adjacent vertebrae. The disclosure further provides a method for implanting the implant.

### Embodiments of the Invention Covering an Implant

A flexible wire structure, connected with a bone anchor at a first end and anulus patch at a second end, is implanted by positioning the implant inside the intervertebral disk space after inserting it through the injured or defective site in the anulus. The flexible wire structure can have various shapes. In a preferred embodiment, it is trumpet or cone-shaped with a hollow interior space, and made of wire mesh or wire weave. The trumpet- or cone-shape is narrow at the first end where it is operably connected with the bone anchor, and open at the second end where it is operably connected with the anulus patch.

The flexible wire structure may also be made of a plurality of wires oriented in substantially the same direction, i.e., running from the first end of the cone and flaring at the second end. It is also within the scope of this disclosure to have a single branch of wire—either a single wire or a plurality of associated wires in a single branch, which can be contained within a tube or other containing structure. A further embodiment contemplated by the disclosure is a flexible wire structure that at a first end is a single branch, which flares out at the second end into a cone that connects with the anulus patch. An additional embodiment is one where the cone- or trumpet-shaped flexible wire structure is made of a spiral of at least one wire.

The wire of the flexible wire structure can be made of nitinol, aluminum, stainless steel, nylon, polypropylene, or another flexible, biocompatible material.

In a preferred embodiment, the bone anchor is a bone screw, connected with the first end of the flexible wire structure. However, other bone anchors are also within the scope of this disclosure. The bone anchor is used to anchor the implant in the intervertebral disk space, after the implant is positioned inside that space so that the second end of the flexible wire structure is sustained in place over the injury or defect in the anulus. A screwdriver can be used to drive a bone screw into the vertebral bone endplate and into the cortical bone. Other varieties of bone anchors will employ other appropriate tools. For the screwdriver or other tool to be able to reach the bone screw or anchor, the anulus patch either can be unattached until after anchoring, or partially attached to allow the tool to reach the bone screw or anchor, or partially folded back.

However, in certain embodiments, it may be possible to pre-attach the anulus patch if the tool used to drive the bone anchor into the vertebral bone endplate can be used without damaging the patch or the rest of the implant. One such embodiment has a single branch of at least one wire that connects with the anulus patch at its second end, i.e., the end distal to the first end that connects with the bone screw.

The anulus patch can be attached all at once after anchoring, or attached partially before anchoring with the remainder waiting until after the anchoring step. The flexible wire structure can engage the anulus patch with hooks at the second end of the implant that are either connected with the wires at the second end, or continuous with them. The hooks further secure the patch and flexible wire structure to the healthy anulus tissue around the injury or defect. Alternatively, the patch can associate with the flexible wire structure via loops connected with the second end of the flexible wire structure which are adapated to receive sutures. The sutures penetrate the anulus patch and the anulus tissue.

The anulus patch can remain on the outside of the anulus, once the implant is positioned with the patch connected with the flexible wire structure. However, a patch that is positioned on the interior wall of the injured or defective anulus is also within the scope of this disclosure. Further, the disclosure contemplates the use of a patch that promotes tissue growth over and around the patch to permanently repair the injury or defect. For instance, the patch can be made of a wire or plastic mesh, or other scarring agent, and/or other appropriate agent that promotes tissue growth.

### Embodiments of the Invention Covering a Method of Implantation

The preferred method of an embodiment of this invention for implanting the prosthetic intervertebral disk implant uses the actual injury site as the point of insertion and positioning of the implant. This approach obviates the need to damage the anulus further with additional incisions for inserting the implant. A cannula with a stylet is first inserted through an incision in the skin. Alternatively, nested cannula can be used, gradually to expand the point of insertion so that the point of insertion is able to accommodate a cannula of sufficiently large diameter to house the implant and any tools necessary in the disclosed method of implantation.

A device, such as an automated Nucleotome® hand-operated tissue cutter, is inserted through the cannula and used to cut and remove any herniated nucleus material. The device is then withdrawn and the implant is placed inside the cannula, with the bone anchor end positioned to be inserted first, followed by the flexible wire structure. A plunger is used to urge the implant through the cannula and through the insertion side. The plunger is withdrawn and a tool, such as a screwdriver is used to drive the bone achor into a vertebral bone endplate. This disclosure contemplates using either the upper or lower bone endplate of two adjacent vertebrae on either side of an injured or defective disk.

Once the screwdriver or other tool is withdrawn, the anulus patch is introduced into the cannula and hooked onto the hooks in the ends of the wires of the flexible wire framework. The hooks are then allowed to engage the healthy anulus tissue around the defect or injury. This is done by removing the cannula used to insert the implant into the invertebral disk space. Alternatively, the anulus patch can be sutured onto the tissue around the injury or defect through loops in the ends of the wires at the second, open end of the flexible wire structure.

Embodiments of this invention further contemplate insertion of a flexible wire structure already fully attached to the anulus patch, so long as a tool can be inserted to drive the bone anchor into the vertebral bone endplate without damaging the anulus patch.

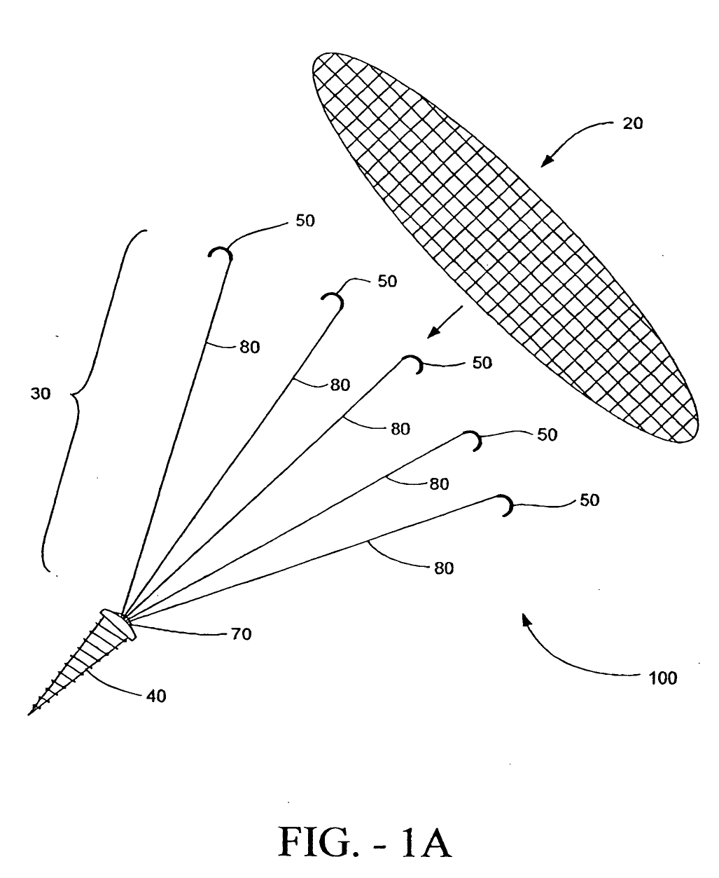

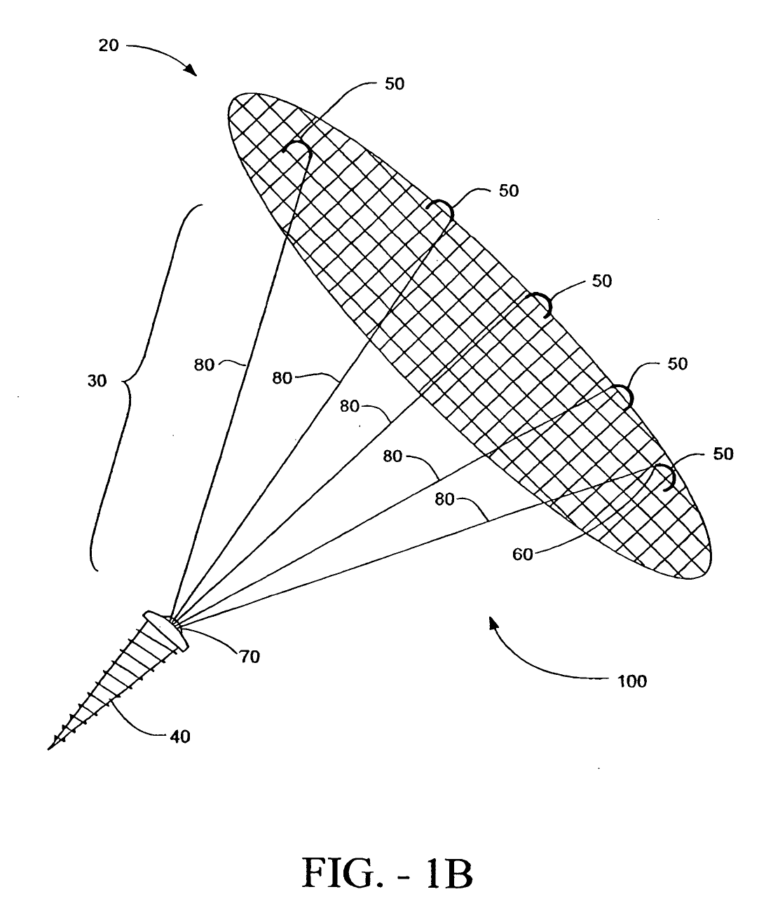

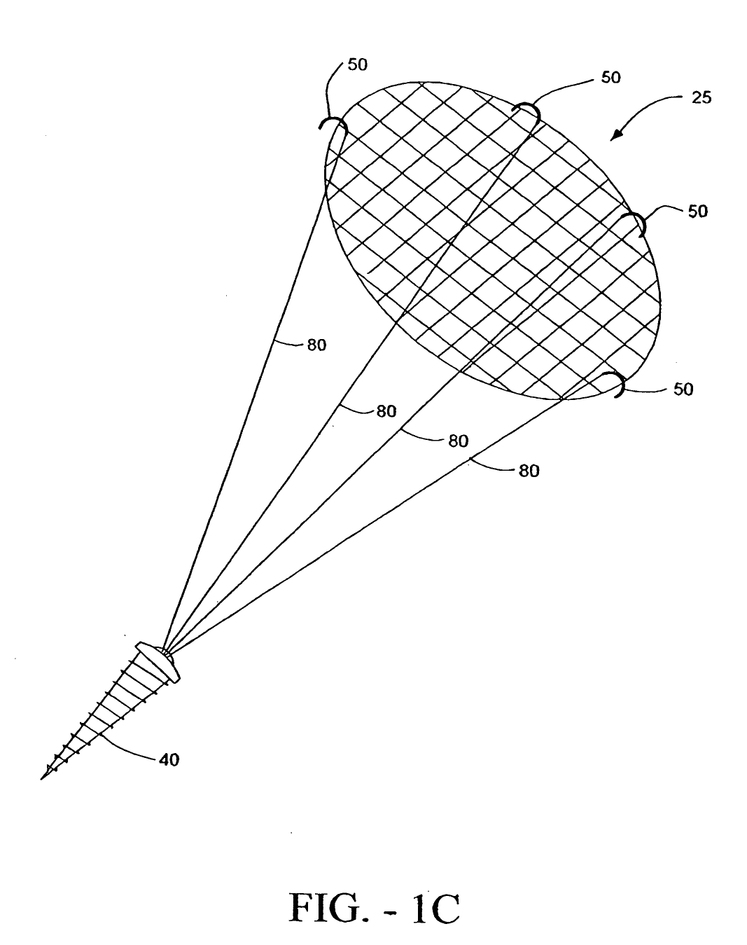

### Embodiments of FIGS. 1A, 1B, and 1C

One preferred embodiment of a prosthetic intervertebral spinal implant for repairing the anulus of an intervertebral disk is shown in FIGS. 1A and 1B. Both figures show a perspective view of an implant with a bone anchor 40, flexible wire structure 30, and anulus patch 20. The bone anchor 40 depicted is a bone screw, but the disclosure encompasses other types of bone anchors including, by way of example only, bone pins and bone sutures that can penetrate the vertebral bone endplate and into the cortical bone. The bone anchor 40 can be made of a biocompatible metal, including nitinol, titanium, and stainless steel.

One purpose of the flexible wire structure 30 is to position and sustain an anulus patch 20 over the injured or defective anulus tissue. A further purpose is to serve as a cushion in the space otherwise occupied by the nucleus pulposus to absorb shocks to the spine and maintain flexibility. The flexible wire structure 30 in both FIGS. 1A and 1B is comprised of wires 80 with a common point of origin which forms the closed end 70 of the cone- or trumpet-shaped structure 30. However, other forms of the flexible wire structure 30 also are within the scope of the disclosure, and are illustrated in additional figures included herein.

At its first closed end 70, the flexible wire structure 30 meets and connects with the bone anchor 40. It is within the scope of this disclosure that the bone anchor 40 can be connected with the flexible wire structure 30 by having the ends of the wires 80 at the first end 70 loop around the head of the bone anchor 40. It should be understood, however, that any connecting means is contemplated by this disclosure to the extent that it allows the bone anchor 40 to be driven into the bone without entangling the flexible wire structure 30 or otherwise interfering with its positioning or damaging its physical integrity.

At the second end 60 of the flexible wire structure 30, the wires 80 flare out relative to the first closed end 70. The wires 80 have hooks 50 extending from the second end 60 of the flexible wire structure 30 that engage the anulus patch 20 and that can engage the healthy tissue surrounding the injury or defect in the anulus. FIG. 1A shows the anulus patch 20 completely separate from the rest of the implant 100. The anulus patch 20 is left off until after the bone anchor 40 has been driven into the vertebral bone endplate, so that the bone anchor can be reached with a tool, such as a screwdriver, without damaging the anulus patch 20. It should be understood, however, that the anulus patch 20 also can be partially attached to several of the hooks 50 before anchoring the implant 10 because the partial attachment would permit use of a tool to drive the bone anchor 40 into the bone without having to puncture or otherwise damage the anulus patch 20. Further, the anulus patch can be fully attached to the structure 30 and then folded back out the way of a tool. FIG. 1B depicts the implant 100 with the anulus patch 20 fully connected with the flexible wire structure 30 with hooks 50 extending from the ends of the wires 80 at the second end 60 of the flexible wire structure 30. FIG. 1C depicts the implant 100 with a plug 25 substituted for an anulus patch 20. The plug 25 serves substantially similar functions as the anulus patch. It can be made of a hydrogel core or cushion contained in a constraining jacket. Alternatively, the constraining jacket can be made of a patient's hair, treated for example as described in Shamie, U.S. Pat. No. 6,416,776. The hydrogel further can contain therapeutic materials. Alternatively, the plug can be made of other appropriate biocompatible material that will remain for a period of time sufficient to ensure promotion of tissue formation over the damage to the anulus. One such example is to use a keratin hydrogel, which has also been described and will not be discussed in detail here.

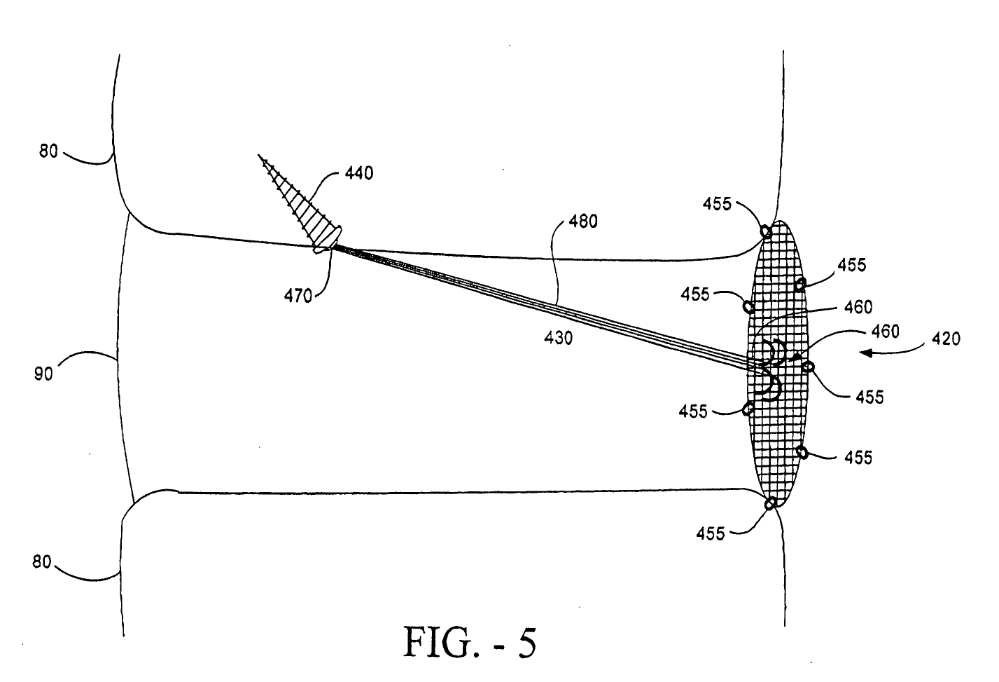

It is also within the scope of the disclosure that the second, open end 60 of the flexible wire structure 30, rather than having hooks 50, would have loops 455 (see FIG. 5). The anulus patch 20 would then be sutured to the healthy tissue around the anulus and also connected with the flexible wire structure 30 through the loops 455 at the second end 60 of the flexible wire structure 30.

The anulus patch 20 is intended to repair damage, such as an injury or defect, to the anulus. It should not only patch the injury or defect, but also promote healing at the site. Patching and scarring can be promoted using a scarring agent, such as wire mesh, plastic mesh, or another inert synthetic mesh of a biocompatible material. Alternatively, a hydrogel plug inside a constraining jacket can be positioned inside the flexible wire structure, with or without a patch over the plug. The hydrogel also can be made of keratin supplied and prepared from the patient's own hair, substantially as described in Zucherman et al., U.S. patent application Ser. No. 10/218,100, which is incorporated herein by reference, or with other hydrogels as taught in the art.

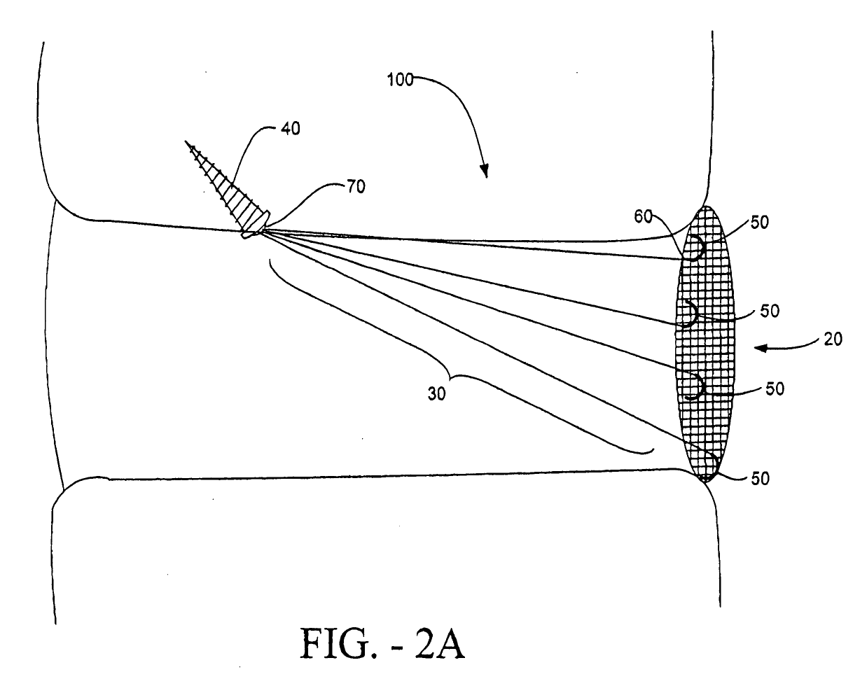

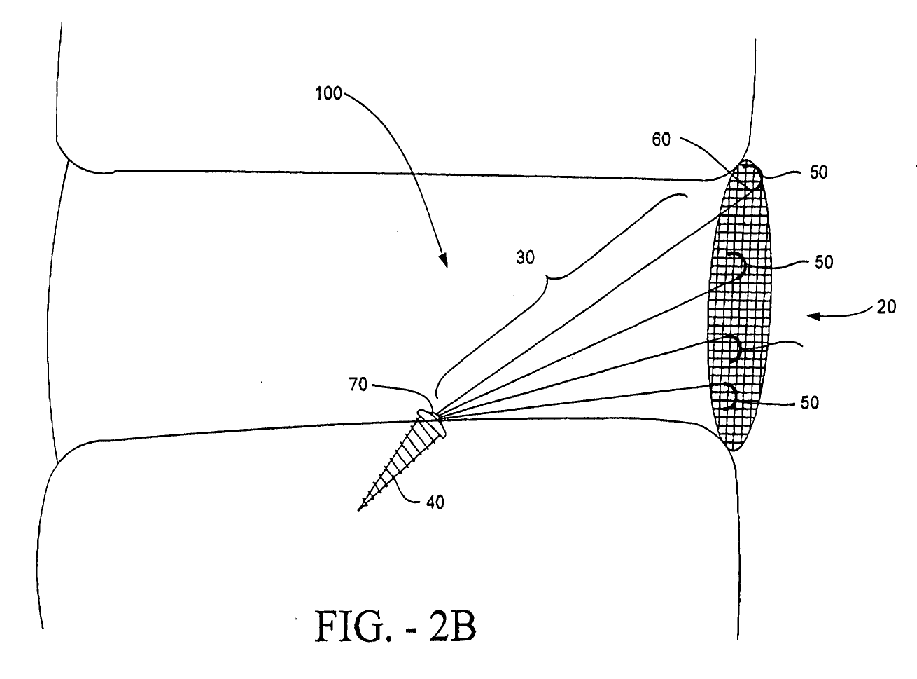

### Embodiments of FIGS. 2A and 2B

As can be seen in FIGS. 2A and 2B, the disclosed implant 100 can be implanted to repair an injury or defect in the anulus of an intervertebral disk 90, by anchoring the implant 100 into either the top (FIG. 2A) or bottom (FIG. 2B) vertebral bone endplate of two adjacent vertebrae 80. It is to be realized that any of the disclosed embodiments to be described herein can be anchored as depicted in FIGS. 2A and 2B.

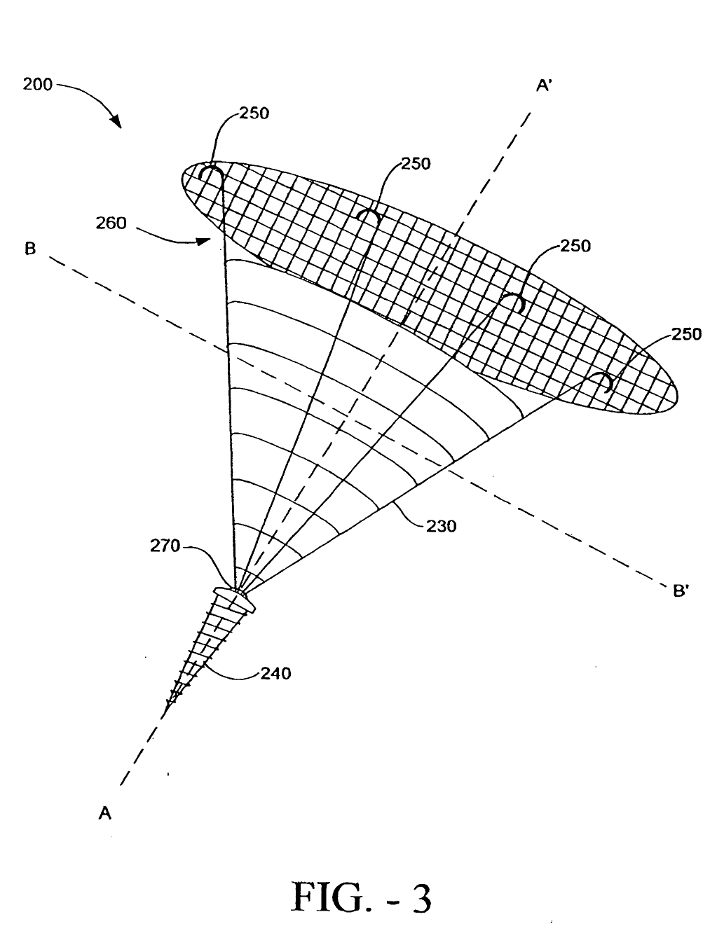

### Embodiment of FIG. 3

A further embodiment 200 is shown in FIG. 3. In this embodiment, the flexible wire structure 230 is composed of a wire mesh or weave, with hooks 250 extending from the second, flared end 260 of the cone- or trumpet-shaped wire structure 230, from the wires extending substantially along the axis defined by A-A′. Alternatively, the hooks 250 need not be continuous with the wires. Instead, they may be fixed separately to the wires or mesh at the open end 260 running substantially along the axis defined by B-B′, at the open end 260 of the flexible wire structure 230. A combination of both types of hooks 250 also is contemplated. Preferably, the wire mesh and the hooks 250 disclosed are made of nitinol, titanium, or stainless steel. They can further be made of nylon, polypropylene, or other flexible biologically inert material.

The hooks 250 engage the anulus patch 220, after the implant 200 is anchored at its first end 270 with a bone anchor 240 to either a first or second vertebral bone endplate, as depicted in FIGS. 2A and 2B. The anulus patch 220 depicted is a mesh. The mesh can either be of wire or plastic, or another inert synthetic biocompatible material that will promote and/or permit tissue growth over the anulus patch 220, or any other material that can have tissue growth-encouraging properties. Alternatively, the anulus patch 220 can be placed over a hydrogel plug encased in a constraining jacket.

The bone anchor 240 depicted in FIG. 3 is a bone screw. However, it is within the scope of this disclosure to employ any type of appropriate bone anchor 240 that can penetrate the vertebral bone endplate and into the cortical bone to anchor the implant 200.

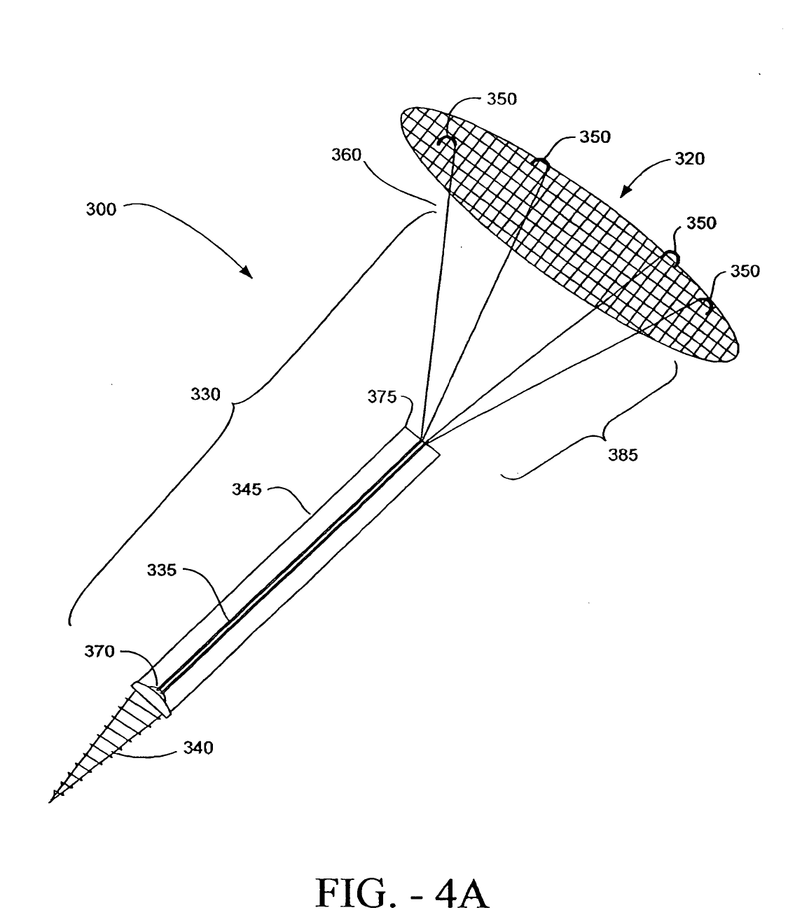

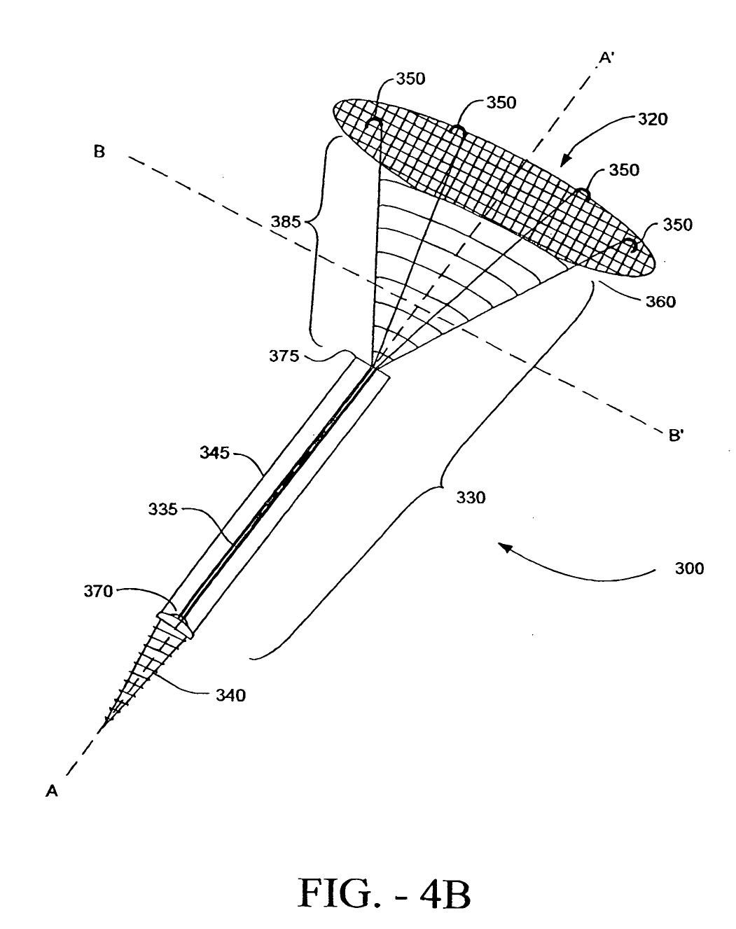

### Embodiment of FIGS. 4A and 4B

A further embodiment is depicted in FIG. 4A. In this embodiment 300, part of the flexible wire structure 330 is made of a plurality of wires associated as a single branch 335 originating at the first end 370 of the flexible wire structure 330. The single branch 335 is depicted as appearing encased inside a tube 345. The tube 345 has its point of origin near the bone anchor 340, where the plurality of wires are connected, and a second end 375 intermediate between the bone anchor 340 and the second, open end 360 of the flexible wire structure 330. Thus, the tube 345 does not run the full length of the flexible wire structure 330, and the plurality of wires emerges where the tube ends 375 to form a cone or mini-trumpet shape 385, as detailed below.

The tube 345 functions to prevent the single branch of wires 335 from fraying or dissociating, and otherwise to protect them. It also serves to brace the single branch of wires 345 so that is sufficiently rigid to sustain the anulus patch 320 in place at the injury or defect in the anulus. The tube 345 can be made of a plurality of flexible biocompatible materials, including plastics and metals such as nitinol, titanium, and stainless steel. It is also within the scope of this disclosure to use fibrous materials and other non-brittle materials for the tube 345. It is also contemplated that the single branch 335 need not be encased by a tube 345.

At or near the point 375 where the single branch of wires 335 emerges from the tube 345, the wires flare out individually to form a cone or mini-trumpet 385 at the second, open end 360 of the flexible wire structure 330, as in FIG. 4A. Alternatively, the wires can be interwoven with wires in a direction substantially perpendicular or at an angle to the direction of the wires emerging from the tube 345, as depicted by axis B-B′ in FIG. 4B. The wires at the open end 360 of the flexible wire structure 330 can have a plurality of hooks 350 adapted to engage the anulus patch 320 with the flexible wire structure 330 and also to engage it with the healthy tissue around the damage, caused by an injury or defect, to the anulus. In the wire weave or mesh depicted in FIG. 4B, the wires in the mini-trumpet 385 can have hooks 350 that are continuous with the wires at the open end 360 of the flexible wire structure 330, and/or the wires substantially parallel or at an angle relative to the axis B-B′ can have separate hooks 350 that are connected with the wires to function as the hooks 350 made from the ends of the wires which are parallel to axis A-A′.

The single branch 335 of the flexible wire structures 330 in FIGS. 4A and 4B, respectively, are connected with a bone anchor 340 at the first end 370, which is depicted in FIGS. 4A and 4B, respectively, as a bone screw. However, as in previous and other embodiments in this disclosure, the bone anchor 340 can be any type of bone anchoring device that can engage the vertebral bone endplate to sustain the implant in position in the intervertebral space.

### Embodiment of FIG. 5

A further embodiment is depicted in FIG. 5. In this embodiment 400, the flexible wire structure 430 is composed of a single branch of at least one wire that extends continuously from the first end 470 of the flexible wire structure 430 that connects with the bone anchor 440 to the second end 460 of the flexible wire structure 430 that connects with the anulus patch 420. The single branch flexible wire structure 430 is adapted to brace the anulus patch 420 and sustain it in place over and around the injury or defect in the anulus. It also maintains the flexibility of the spine and cushions the shock to the spine.

The single branch of the flexible wire structure 430 can either be a single thick wire, or a plurality of wires that are associated or woven together as a branch. The flexible wire structure 430, whether made of a single wire or a plurality of wires, can be encased in a tube (not shown) that is substantially similar to the tube 345 in embodiment 300 depicted in FIGS. 4A and 4B.

The flexible wire structure 430 connects with the anulus patch 420 at the second end 460 of the flexible wire structure 430, which second end is distal from the first end 470 that connects with the bone anchor 440. The connection between the second end 460 and the anulus patch 420 can be made using a plurality of hooks 450 at the second end 460, which extend from the second end 460 of at least one of the wires. The hooks 450 should pierce the anulus patch 420 at an area substantially at its center, and emerge through to the side of the anulus patch 420 facing out from the intervertebral disk. The hooks 450 should then be made to puncture the anulus patch 420 near the site from where it emerged, to emerge again on the side facing the intervertebral disk space.

It should be understood that the means for connecting the anulus patch 420 with the single branch of the flexible wire structure 430 may also include means that do not require piercing the anulus patch 420. By way of example only, the wires in the single branch of the flexible wire structure 430 may be unraveled near the second end 460, and then flared out in a plane that is substantially parallel to the faces of the anulus patch 420, forming a plate that can be adhered to the anulus patch 420 using a biocompatible adhesive or using biocompatible ties. The single branch of the flexible wire structure 430 also may be sutured to the anulus patch 420, or woven into the anulus patch 420.

A plurality of sutures 445 can be used to engage the anulus patch 420 with the anulus tissue surrounding the damaged site. In this embodiment, the sutures do not engage the tissue and anulus patch 420 with the flexible wire structure 430. Rather, the sutures only connect the anulus patch 420 with the anulus tissue. The flexible wire structure 430 sustains the patch in place only from inside the intervertebral disk space, unlike other embodiments described here, where the flexible wire structure 430 also is used to engage the anulus patch 420 with the anulus tissue.

As in the other embodiments, the bone anchor 440 depicted is a bone screw, but other bone anchors 440 are also within the scope of the disclosure. Also, the anulus patch 420 can be composed, if desired, of any of a wire mesh, a plastic mesh or other scarring agent made from an inert synthetic biocompatible material, adapted to promote growth of scar tissue around and over the anulus patch so that the result is sealing of the damage to the anulus resulting from an injury or defect.

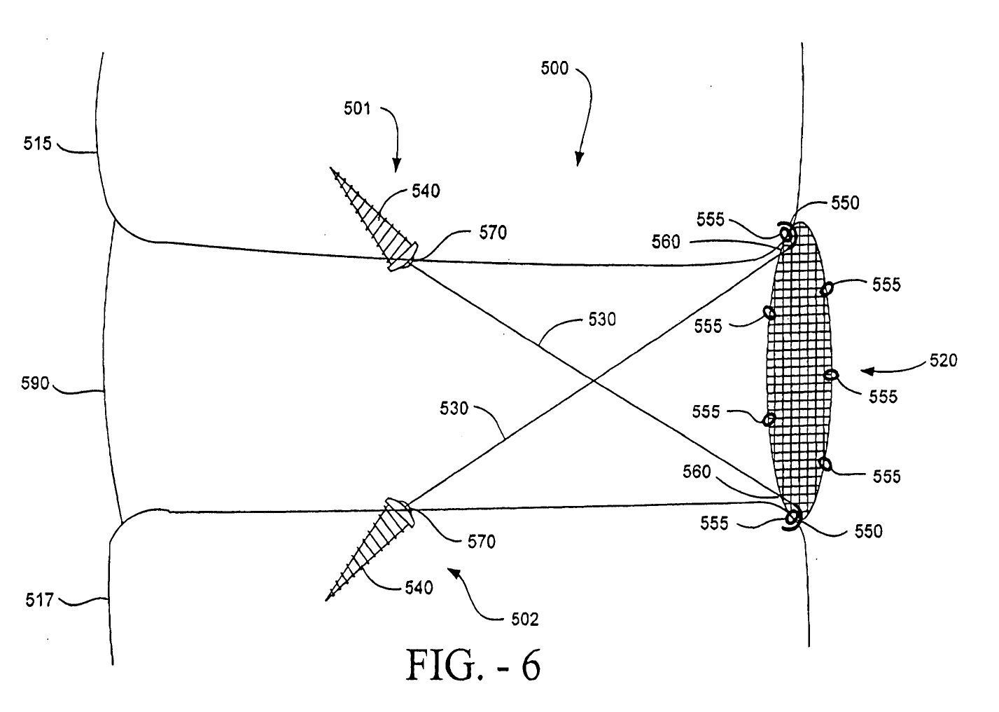

### Embodiment of FIG. 6

A further embodiment of the disclosure is depicted in FIG. 6. In this embodiment 500, two implants 501 and 502, which can be any of the embodiments described above, are used from within the intervertebral disk space to brace an anulus patch 520 and sustain it in place over a defect or injury to the anulus, while also cushioning impact on the spine. A first implant 501 is anchored to a first vertebral bone endplate 515, and a second implant 502 is anchored to a second vertebral bone endplate 517.

Essentially, two flexible wire structures 530, one from each implant 501 and 502 connect with a single anulus patch 520 that seals a damaged site in the anulus. A plurality of hooks 550 extend from the second ends 560 of the two flexible wire structures 530 and connect with the anulus. Alternatively, loops 555 may be formed from or attached to the second ends 560 of the flexible wire structures 530 and adapted to receive sutures to engage the anulus patch 520 with the anulus.

As with other embodiments, the bone anchor 540 is depicted as a bone screw, but other bone anchors 540 are within the scope of the disclosure. The anulus patch 520 also has been described in other embodiments.

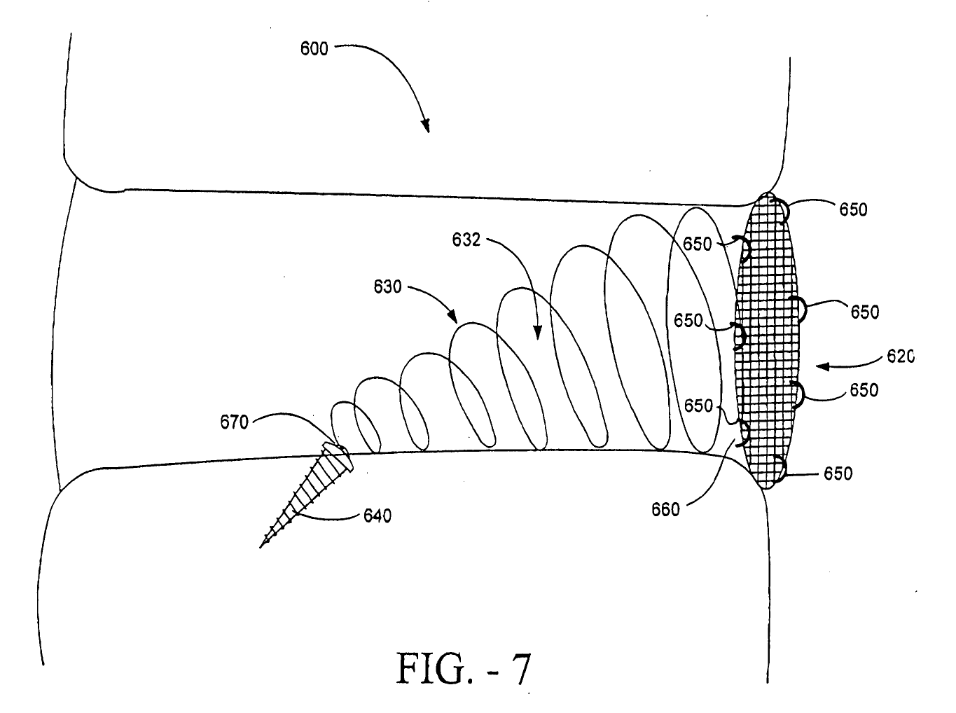

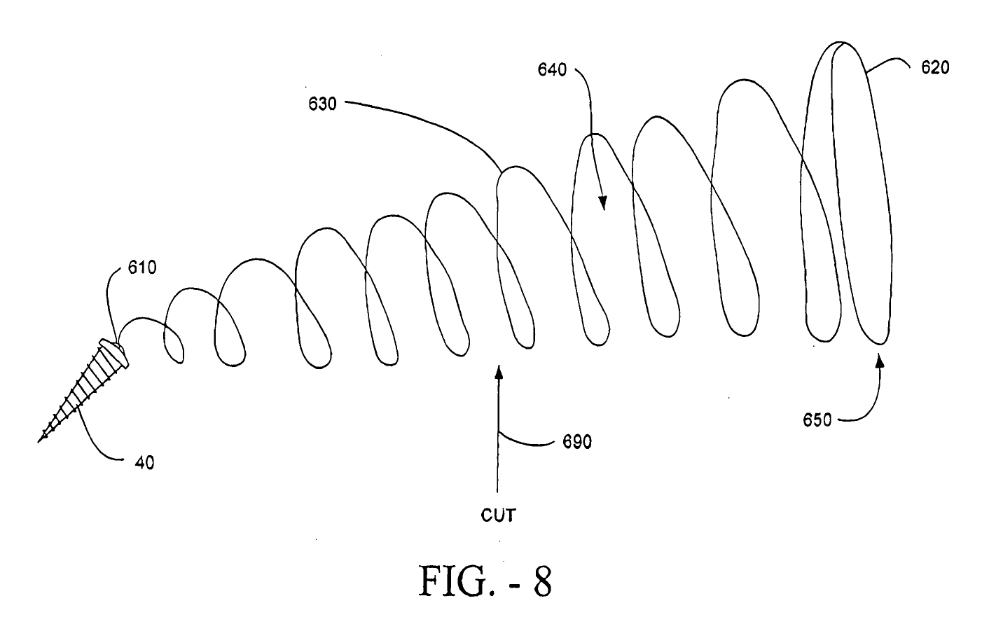

### Embodiment of FIGS. 7 and 8

A further embodiment is depicted in FIGS. 7 and 8. In this embodiment 600, the flexible wire structure 630 is in the shape of a spiral cage with a hollow interior space 632. The spiral cage wire structure 630 is open at its second end 660 and is substantially closed at its first end 670, substantially similar to the cone- or trumpet-shape of other embodiments already described. The spiral cage wire structure 630 comprises at least one wire that is connected at the first end 660 with a bone anchor 640, and is adapted to connect, after anchoring, with an anulus patch 620.

The spiral cage 630 connects with the anulus patch 620 by a plurality of hooks 650 that are adapted to connect with the rim of the open end 620 of the spiral cage 630 and with the periphery of the anulus patch 620 and the tissue surrounding the defect in the anulus. The anulus patch 620 can be connected completely with the spiral cage 630 and positioned over and around the injury or defect in the anulus after the implant 600 is anchored in the vertebral bone endplate. The anulus patch 620 can remain unattached entirely until after anchoring. Alternatively, the anulus patch 620 can initially be partially attached to the hooks 650 on the rim of the spiral cage 630 before anchoring and, once any tools needed for engaging the bone anchor 640 with the vertebral bone endplate are removed from between the vertebrae, the anulus patch 620 can be fully connected with the rim of the spiral cage wire structure 630.

It is to be realized that other means of engaging the anulus patch 620 with the spiral cage 630 are also within the scope of the disclosure. By way of example only, wire loops 555 (shown in FIGS. 5 AND 6) can be used to connect the rim of the spiral cage 630 with the anulus patch 620. Such loops 555 are adapted to receive sutures that will engage the anulus patch 20 with the healthy tissue around the defect or injury to the anulus.

FIG. 8 shows that this spiral wire structure 630 can be adjusted to accommodate intervertebral disk space anatomy of different dimensions. The physician can use known means for measuring the dimensions of the patient's intervertebral disk space and determine the proper dimensions for an implant as disclosed herein. The physician can then adjust the size of the implant 600 accordingly by cutting the implant 600 as depicted 690 in FIG. 8.

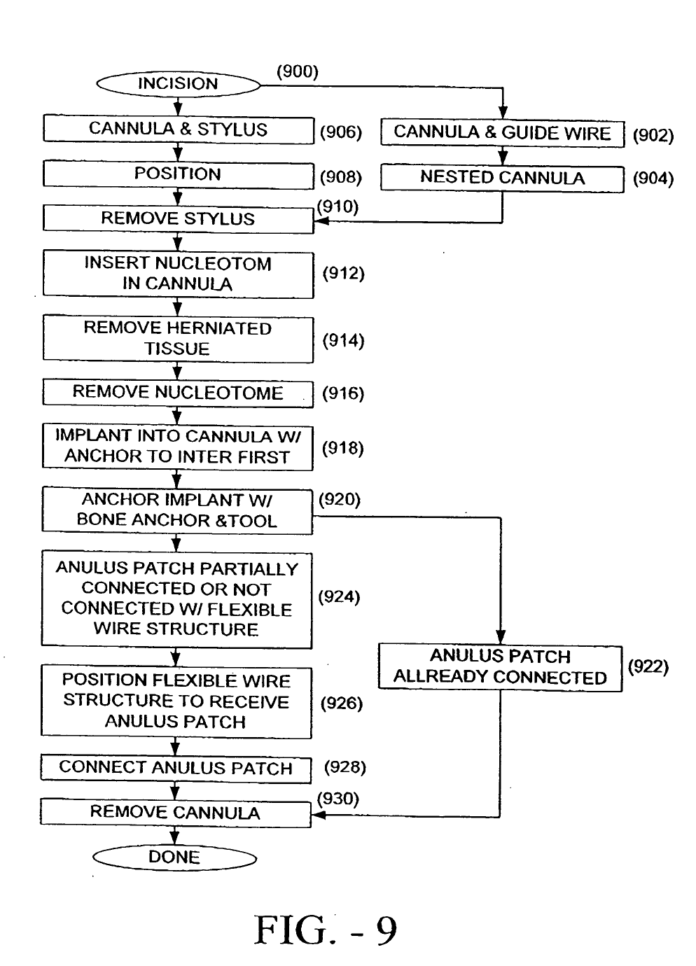

### Embodiment of FIG. 9

An embodiment of a method for implanting a spinal disk repair implant is depicted in flow chart format in FIG. 9. First, an incision or puncture is made using a posterior approach 900. A cannula is inserted with a stylus 906 and the cannula moved into position at the site of the injury or defect to the anulus 908. The stylus is then removed 910, and a Nucleotome® tool is inserted into the cannula 912. The Nucleotome® tool includes a guillotine blade that can be used to excise herniated nucleus pulposus material 914.

As an alternative to a cannula/stylus, nested cannulae and a guidewire 902 can be used to position the cannulae and widen gradually the incision and to access the intervertebral disk space. The guidewire is inserted first, followed by successively wider-bore cannulae 904. The smaller interior cannulae are then removed, as well as the guidewire, and a larger operating space is available through the broadest cannula. The Nuceotome is then inserted 912 and applied to remove herniated disk material, as above 914.

Once the herniated nucleus material is removed, the Nucleotome® tool is extracted from the cannula 916 and the implant can be anchored. An implant essentially as described above is inserted into the cannula with the bone anchor inserted first 918, so that the bone anchor is the first part to penetrate through the defect in the anulus that is to be repaired. Inserting the implant through the same part of the anulus that already has been damaged is beneficial to the patient, since it may avoid further injury to the anulus, which may result from making additional incisions in it. For example, cutting flaps out of the anulus could cause a loss of integrity of its fibrous layers. Further injury may result from any weakening in the anulus, and the possibility of its healing completely would be reduced.

The implant with the anulus patch pre-attached 922 can be urged down the cannula toward the injury or defect using an instrument that serves as a plunger. Alternatively, it can be moved through the cannula with a rigid tool to push the implant along and maintaining the proper orientation—bone anchor first—as it travels down the cannula. The plunger or other tool is then removed.

Once the bone anchor is in the intervertebral disk space, a tool is inserted into the cannula to cause the bone anchor to engage with a vertebral bone endplate 920. If the bone anchor is a bone screw, then the tool is a screw driver with a head adapted to engage the bone screw and drive it into the bone endplate. Alternatively, the bone anchor can be a type of anchor or bone suture that is able to penetrate the bone endplate.

After the bone anchor is engaged, the tool used to drive the bone anchor into the bone endplate is removed from the cannula. The flexible wire structure of the implant is left in position to receive the anulus patch at the site of the injury or defect to the anulus 926.

In certain embodiments, the anulus patch is attached completely at this point 922. As described for the various embodiments, the connecting means can be hooks at or near the ends of the wires at the second end of the flexible wire structure, or loops that can receive sutures to engage the anulus patch with the healthy tissue around the defect or injury in the anulus and with the flexible wire structure.

Other embodiments will require the anulus patch to be attached, either partially or entirely, after anchoring the implant in the intervertebral space 924. At this point, hooks or loops and/or sutures are used to make this connection 928. It is within the scope of this disclosure for a tool, such as a small forceps or other effective tool, to be used to position the anulus patch and pierce it with the hooks that are to hold it in position and engage the anulus tissue around the defect or injury. Alternatively, the forceps or other effective tool can be used to manipulate loops into position to receive sutures that will hold the patch to the anulus and the flexible wire structure.

Alternatively, an anulus patch fully attached to the second end of the flexible wire structure can spring out of the end of the cannula prior to the bone screw being attached to the bone. The bone screw can then be used to secure the flexible wire structure to the vertebra. Thereafter, the anulus patch and hook of the flexible wire structure can be manipulated into engaging with the tissue surrounding the tear in the anulus.

The cannula is then removed from the incision and the incision is surgically closed 930.

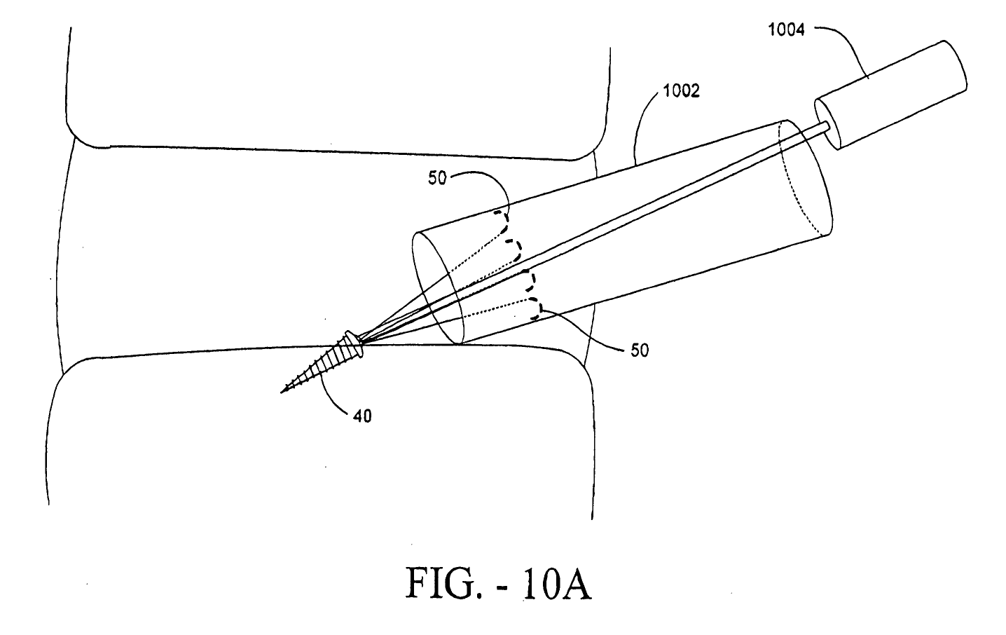

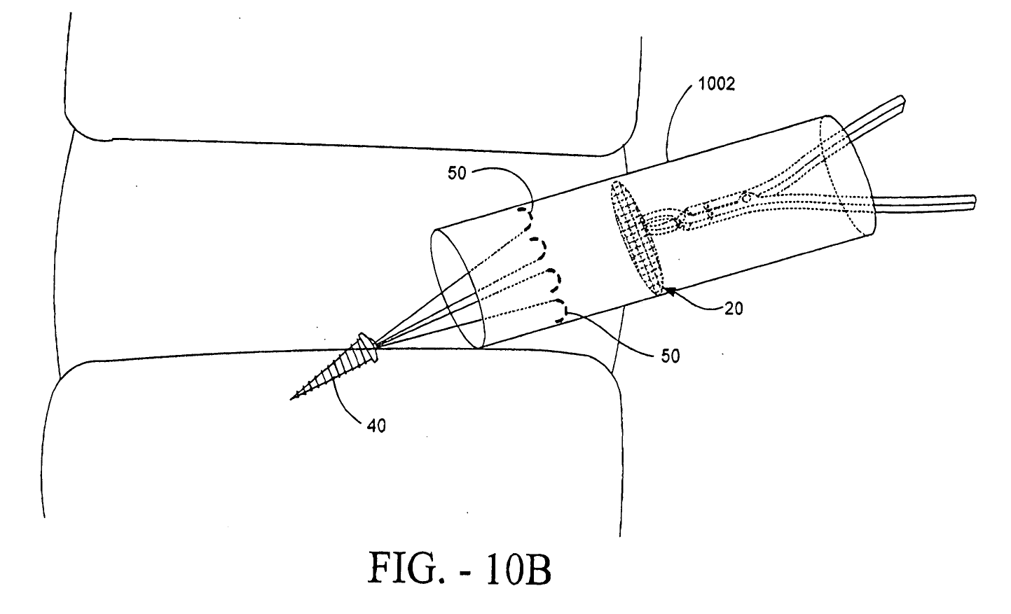

### Embodiment of FIG. 10

An embodiment of a method for implanting a spinal disk repair implant is depicted in FIG. 10. In this embodiment, as depicted in flowchart format in FIG. 9, a cannula 1002 is inserted toward the damaged site on the annulus and the implant is inserted with the bone anchor 40 end first. The implant is pushed through the cannula 1002 and toward the damaged site. The anchor is engaged using an appropriate tool. Here, a bone screw is depicted and the appropriate tool is a screw driver 1004. Other tools and bone anchoring devices are within the scope of this disclosure.

Once the implant is anchored, an anulus patch 20 is engaged with the flexible wire structure and the tissue surrounding the damaged site on the anulus, using methods described in detail above. If the anulus patch 20 already is attached to the flexible wire structure, as with embodiments having a flexible wire structure comprising a single branch of wires or a wire, then all that remains is to ensure that the connecting means for engaging the patch further are manipulated to engage the tissue of the anulus surrounding the damaged site.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its pratical application, thereby enabling others skilled in the art to understand the invention and the various embodiments and with various modifications that are suited to the particular use contemplated. It is intended that the scope of the invention be defined by the following claims and its equivalence.

1. An implant for repairing damage to an anulus of an intervertebral disk, the implant comprising:
an first anchor part, adapted to anchor the implant to a first vertebra from within the intervertebral disk space;
a second structure part with a first end and a second end attached to the first anchor part; and
a third patch part that connects with the second end of the second structure part, adapted to patch the damage to the anulus.

2. The implant of claim 1 wherein the first anchor part is a bone anchor.

3. The implant of claim 2 wherein the bone anchor is made of a material selected from the group consisting of nitinol, titanium, stainless steel and a resorbable material.

4. The implant of claim 1 wherein the second structure part has a conical shape with a narrow end and an open end.

5. The implant of claim 4 wherein the second structure part comprises a plurality of wires.

6. The implant of claim 1 wherein the second structure part is made of a material selected from the group consisting of nitinol, titanium, and stainless steel.

7. The implant of claim 4 wherein the second structure part comprises a wire mesh.

8. The implant of claim 1 wherein two such implants are used within the same intervertebral disk space, a first implant anchored by its first anchor part in a first vertebrae and a second implant anchored by its first anchor part in a second vertebrae.

9. The implant of claim 4 wherein the second structure part comprises a plurality of wires wound as a spiral.

10. The implant of claim 9 wherein the second structure part spiral can be shortened from a longer length to accommodate different the anatomy of the intervertebral disk space.

11. The implant of claim 1 wherein the third patch part is made of scarring agents selected from the group consisting of wire mesh, plastic mesh, inert synthetic biocompatible materials, and structural filaments.

12. The implant of claim 1 wherein the third patch part is placed over a hydrogel plug encased in a constraining jacket.

13. The implant of claim 12 wherein the hydrogel plug contains therapeutic agents.

14. An implant for repairing intervertebral disks, the implant comprising:
a bone anchor adapted to engage a vertebra within the intervertebral disk space;
a flexible wire structure having a first end and a second end, the first end connected with the bone anchor; and
an anulus patch that connects with the second end of the flexible wire structure, adapted to repair damage or injury to the anulus, while promoting healing.

15. The implant of claim 14 wherein the anulus patch is made of a scarring agent selected from the group consisting of plastic mesh, wire mesh, inert synthetic biocompatible materials, and structural filaments.

16. The implant of claim 14 wherein the anulus patch is placed over a hydrogel plug encased in a constraining jacket.

17. The implant of claim 16 wherein the hydrogel plug contains therapeutic agents.

18. The implant of claim 1 wherein the size of the second structure part can be adjusted to accommodate the anatomy of an intervertebral disk space.

19. A method for repairing a defect in an anulus of an intervertebral disk comprising:
making a surgical incision to expose the defect in the anulus;
inserting a cannula adjacent to the defect;
excising herniated disk tissue if necessary;
positioning an implant in the cannula;
urging the implant through the cannula and into the intervertebral disk space;
anchoring the implant to a vertebra within the intervertebral disk space; and
positioning a patch to seal the damaged site of the anulus;
securing the patch to the anulus.

20. The method of claim 19 wherein the implant has an anchor means at one end and is positioned in the cannula so that the anchor means enters the damaged site first.
